(19) United States
(12) Patent Application Publication
(10) Pub. No.: US 2010/0074392 A1
(43) Pub. Date: Mar. 25, 2010

(54) X-RAY TUBE WITH MULTIPLE ELECTRON SOURCES AND COMMON ELECTRON DEFLECTION UNIT

(75) Inventors: Rolf Karlotto Behling, Norderstedt (DE); Gerald James Carlson, Aurora, IL (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/517,216

(22) PCT Filed: Nov. 30, 2007
(86) PCT No.: PCT/IB07/54867

§ 371 Date: Jun. 2, 2009

(30) Foreign Application Priority Data
Dec. 4, 2006 (EP) 06125334.0

(51) Int. Cl. H01J 35/30; H01J 35/14; H01J 35/08; A61B 6/00

(52) U.S. Cl. 378/4; 378/137; 378/124

(57) ABSTRACT

It is described an X-ray tube (100, 200) for moving a focal spot within a wide range. The X-ray tube (100, 200) comprises a first electron source (105), which is adapted to generate a first electron beam projecting along a first beam path (107a, 107b), a second electron source (110), which is adapted to generate a second electron beam projecting along a second beam path (112a, 112b) and an anode (120), which is arranged within the first beam path (107a, 107b) and within the second beam path (112a, 112b) such that on a surface (121) of the anode (120) the first electron beam (307a) generates a first focal spot (308) and the second electron beam (412a) generates a second focal spot (413). The X-ray tube (100, 200) further comprises a common deflection unit (130, 330, 430), which is adapted to deflect the first (307a) and the second electron beam (412a), such that the positions of the first (308) and the second focal spot (413) is shifted. The electron sources (105, 110) may be arranged within a linear array allowing for a simple mechanical support of the X-ray sources.

### FIELD OF INVENTION

The present invention relates to the field of generating X-rays by means of X-ray tubes. In particular, the present invention relates to an X-ray tube, which is adapted to generate at least two X-ray beams originating from at least two different focal spot positions. Thereby, the at least two X-ray beams may be activated simultaneously or preferably in an alternating manner. Such types of X-ray tubes are called multiple focal spot X-ray tubes.

The present invention further relates to an X-ray system, in particular to a medical X-ray imaging system, wherein the X-ray system comprises an X-ray tube as mentioned above.

Further, the present invention relates to a method for generating X-rays, which are in particular used for medical X-ray imaging. The X-rays are generated by means of an X-ray tube as mentioned above.

### ART BACKGROUND

Computed tomography (CT) is a standard imaging technique for radiology diagnosis. However, the use of an X-ray tube comprising only a single focal spot sometimes causes reconstruction problems in particular when large objects have to be examined. Thereby, for a certain viewing angle border regions of the object may not be located within the X-ray beam originating from the single focal spot and impinging onto the detector. This has the effect that for these border regions only a reduced number of projection views are available such that the quality of the three-dimensional (3D) reconstruction of the object under examination is reduced. In particular, reconstruction artifacts may be generated, which erroneously indicate structures, which are in reality not existent.

In order to increase the available number of projection views also for border regions, dual focus spot X-ray tubes can be used. Thereby, for each viewing angle of a CT scanning unit, which comprises the X-ray source and the X-ray detector, two two-dimensional (2D) X-ray attenuation datasets representing two different projection angles can be generated. Of course, the spatial distance between the two focal spots defines the angular difference between these two 2D X-ray attenuation datasets. Thus, a large spatial distance between the two focal spots is advantageous in many applications.

U.S. Pat. No. 6,125,167 discloses a rotating anode X-ray tube with multiple simultaneously emitting focal spots. The X-ray tube includes a body defining a vacuum envelope. A plurality of anode elements each defining a target face are rotatably disposed within the vacuum envelope. Mounted within the vacuum envelope, a plurality of cathode assemblies are each capable of generating an electron stream toward an associated target face. A filament current supply applies a current to each of the cathode assemblies, and is selectively controlled by a cathode controller, which powers sets of the cathodes based on thermal loading conditions and a desired imaging profile. A collimator is adjacent to the body and defines a series of alternating openings and septa for forming a corresponding series of parallel, fan-shaped x-ray beams or slices.

US 2006/0104418 A1 discloses a wide scanning imaging X-ray tube. The imaging tube includes a cathode that emits an electron beam and an anode. The anode includes multiple target surfaces. Each of the target surfaces has a focal spot that receives the electron beam. The target surfaces generate multiple x-ray beams in response to the electron beam impinging on the target surfaces. Each x-ray beam is associated with one of the target surfaces. An x-ray imaging system includes the cathode and the anode. A controller is electrically coupled to the cathode and adjusts emission of the electron beam on the anode.

US 2006/0018432 A1 discloses a large-area individually addressable multi-beam X-ray system. The multi-beam X-ray system has a plurality of stationary and individually electrically addressable field emissive electron sources with a substrate composed of a field emissive material, such as carbon nanotubes. Electrically switching the field emissive electron sources at a predetermined frequency field emits electrons in a programmable sequence toward an incidence point on a target. The generated X-rays correspond in frequency and in position to that of the field emissive electron source. The large-area target and array or matrix of emitters can image objects from different positions and/or angles without moving the object or the structure and can produce a three dimensional reconstructed image. The X-ray system is suitable for a variety of applications including industrial inspection, quality control, analytical instrumentation, security systems such as airport security inspection systems, and medical imaging, such as computed tomography.

There may be a need for providing a multiple beam X-ray tube, which allows for an easy and reliable focusing of the different electron beams being assigned to different focal spot positions.

### SUMMARY OF THE INVENTION

This need may be met by the subject matter according to the independent claims. Advantageous embodiments of the present invention are described by the dependent claims.

According to a first aspect of the invention there is provided an X-ray tube. The provided X-ray tube comprises (a) a first electron source, which is adapted to generate a first electron beam projecting along a first beam path, (b) a second electron source, which is adapted to generate a second electron beam projecting along a second beam path and (c) an anode, which is arranged within the first beam path and within the second beam path. Thereby, on a surface of the anode the first electron beam generates a first focal spot and the second electron beam generates a second focal spot being separated from the first focal spot. The provided X-ray tube further comprises a common deflection unit, which is adapted to deflect the first electron beam and the second electron beam, such that the position of the first focal spot and the position of the second focal spot is shifted.

This aspect of the invention is based on the idea that it is not necessary to provide one deflection unit for each electron beam. It is rather possible to use a common deflection unit both for the first electron beam and for the second electron beam. This may provide the advantage that the provided dual electron source X-ray tube can be realized in a mechanical comparatively simple design such that the manufacturing expenses can be kept low. Further, the provision of only one deflection unit being assigned to both electron sources may comprise the advantage, that compared to the provision of two individual deflection units it is easier to find an arrangement where the deflection unit is located so that it does not interfere with the x-ray beams originating from both the first and second focal spots.

It has to be mentioned that when the two electron beams are generated in an alternating manner, the common deflection unit may allow for an individual deflection of both the first electron beam and the second electron beam. Thereby, the common deflection unit may be operated in a synchronized manner with respect to the switching frequency of the two electron beams.

Compared to X-ray tubes having a single electron source only, the distance from the electron emitters of the individual electron sources to the target position on the anode surface can be kept much smaller. This may allow for a high electron beam current density and makes the focusing of the corresponding electron beam much easier.

According to an embodiment of the present invention the X-ray tube further comprises a control unit, which is coupled to the first electron source, to the second electron source and to the common deflection unit. The control unit is adapted to control the first electron source, the second electron source and the common deflection unit in a synchronized manner. This may provide the advantage that the emission of the first electron beam and the second electron beam and the operation of the common deflection unit can be controlled in such a manner that a timed sequence of various beam deflections is accomplished in accordance with a timed sequence of electron beam generations.

According to a further embodiment of the invention the anode comprises a first focal spot region and a second focal spot region being at least partially separated from the first focal spot region. Thereby, the first focal spot region is assigned to the first electron source and the second focal spot region is assigned to the second electron source. This means that the first focal spot is generated within the first focal spot region and the second focal spot is generated within the second focal spot region, respectively.

It has to be mentioned that the different focal spot regions can be completely separated from each other. This means that there is no overlap between the first and the second focal spot region and the position of the electron spot can be moved over the anode surface in a discrete manner only. Alternatively, neighboring focal spot regions may have an overlap with each other or they may directly border with each other.

In case the electron sources are operated in a synchronized manner such that alternating electron beams are generated, this may allow for an effective quasi-continuous focal spot shift over different focal spot regions. Thereby, the focal spot can be shifted along a comparatively long distance, wherein by contrast to a large focal shift of a single electron beam only, the beam paths are much shorter. Thus, defocusing and other deteriorating effects regarding the quality of the electron beam can be kept within small limits.

By controlling the electron emission from the various electron sources the intensity of the corresponding electron beam and, as a consequence, also the intensity of the corresponding X-ray beam can be controlled very easily.

According to a further embodiment of the invention the first electron source is adapted to activate and to deactivate the first electron beam and/or the second electron source is adapted to activate and to deactivate the second electron beam. Such a switching of the electron beams can be accomplished preferably by applying an electrostatic field close to the electron emitter, which typically is a hot cathode. Thereby, an electrostatic force is acting on electrons, which just have been released from the electron emitter and which represent a space charge cloud surrounding the electron emitter. By varying this electrostatic field the number of electrons can be controlled, which electrons are leaving this electron cloud and which electrons are propagating to the anode. By discretely switching the electrostatic force the electrons being present in the electron cloud surrounding the electron emitter are removed from the cloud in a pulsed manner. Thereby a pulsed electron beam can be generated.

The described electrostatic force acting on the electrons can be generated by means of a grid being arranged close to the electron emitter. Such a grid, which allows to precisely control the electrostatic field at the position of the electron emitter, can be penetrated by the electrons leaving the electron source and being directed to the anode. Thereby, the grid does not spatially inhibit the electron beam propagation.

According to a further embodiment of the invention the common deflection unit is a magnetic deflection unit. Thereby, the strength of the electron beam deflection and, as a consequence, the point of incidence on the anode target i.e. the position of the focal spot can be controlled easily by the strength of the magnetic field. Preferably, the magnetic field covers not only a limited spatial region between the anode and the various electron sources, the magnetic field may rather also cover a region surrounding the electron sources. Thereby, the size of the interaction region of (a) the magnetic deflection unit and (b) the electron beams can be maximized. As a consequence the achievable deflection angle respectively the length of the focal spot shift can be increased.

It has to be mentioned that a coil respectively a solenoid generating the magnetic field should be designed in such a manner that that Eddy currents, which might distort the homogeneity of the magnetic field, are limited to small currents as far as possible. In particular, Eddy currents arising during a transition between a first time period used for deflecting the first electron beam and a second time period used for deflecting the second electron beam should be minimized.

According to a further embodiment of the invention the magnetic deflection unit is adapted to generate a homogeneous magnetic field having a uniform magnetic field intensity at least within a region covering at least partially the first beam path and the second beam path. This makes the mechanical design and electrical supply of the common deflection unit comparatively easy.

The homogeneous magnetic field can be generated for instance by means of a magnetic double yoke in connection with a solenoid being attached to the double yoke. Thereby, the magnetic double yoke comprises two elongated yokes, which define a spatial region exhibiting a homogeneous magnetic field. Thereby, the electron beams pass through this spatial region over at least part of the distance from the electron source to the anode.

It has to be mentioned that when using a magnetic double yoke it is advantageous for a maximal homogeneity of the magnetic field not to magnetically saturate the magnetic material of the yokes. Thereby, a linear relationship between the current powering the solenoid and the magnetic field extending between the yokes can be maintained.

According to a further embodiment of the invention the first electron source and/or the second electron source is made from a non-ferromagnetic material.

This may provide the advantage that the magnetic field can penetrate into the electron sources such that the magnetic field can be kept homogenous along the full first beam path and the second beam path.

According to a further embodiment of the invention the X-ray tube further comprises a further electron source, which is adapted to generate a further electron beam projecting along a further beam path. Thereby, the further electron beam generates a further focal spot on the surface of the anode, wherein the further focal spot is separated from the first focal spot and from the second focal spot. The common deflection unit is adapted to deflect the further electron beam such that the position of the further focal spot is shifted.

It has to be mentioned that in principle the described X-ray tube can be provided with an infinite number of electron sources. Of course, the further electron source may be designed according to any one of the embodiments described above and as will be described below.

According to a further embodiment of the invention the first electron source, the second electron source and the further electron source are arranged in a linear array of electron sources. This may provide the advantage that all electron sources can be mechanically supported by means of a comparatively simple attachment system. Further, the electron sources can be positioned with respect to the anode in a collision free arrangement. This means that neither the electron sources nor the attachment system for the electron sources shadows any one of the X-ray beams originating from the various focal spots.

According to a further embodiment of the invention the anode comprises a flat anode surface at least along a direction being defined by the various focal spot positions. This may provide the advantage that each focal spot can be shifted continuously over the anode surface. Thereby, the relevant topology of the anode surface makes it easy to shift the focal spot with a velocity, which is determined predominately by the derivative with respect to time of a magnetic field deflecting the corresponding electron beam.

It has to be mentioned that different types of anodes can be used. In particular the flat anode can be either a rotatable anode or a stationary anode.

According to a further embodiment of the invention (a) the control unit is adapted to control the electron sources such that the first electron beam and the second electron beam are generated in an alternating manner and (b) the control unit is further adapted to control the common deflection unit in a synchronized manner with respect to the control of the electron sources such that there is produced a quasi-continuous shift of an active focal spot. Thereby, within a first time period the first focal spot represents the active focal spot and within a second time period the second focal spot represents the active focal spot, respectively.

This means that the quasi-continuous focal spot shift can be accomplished along a comparatively long distance covering different focal spot regions. Thereby, as described already above, each focal spot region is assigned to one electron source. Therefore, depending on the number of employed electron sources the focal spot shift can be much larger as compared to a focal spot shift, which would be achievable with single electron source X-ray tube.

When a magnetic deflection unit is used the corresponding varying magnetic induction may be generated by means of a solenoid, which is powered by an alternating current.

According to a further embodiment of the invention the anode comprises a structured anode surface at least along a direction being defined by the various focal spot positions. This may provide the advantage that for different predefined positions of focal spots the geometry respectively the contour of the anode surface can be adapted in order to optimize the anode topology for the corresponding X-rays originating from the different focal spots. Thereby, one or more predefined positions can be assigned to one electron source.

The structured anode can be for example a stacked anode comprising a plurality of anode portions, which can be designed in a modular way. This may provide the advantage that when manufacturing the X-ray tube the structured anode can easily be adapted to the number of electron sources. The structured anode can also comprise a variety of different anode blades extending along a circumference of the anode in a radial direction.

It has to be mentioned that different types of anodes can be used. In particular the structured anode can be either a rotatable anode or a stationary anode.

According to a further embodiment of the invention (a) the control unit is adapted to control the electron sources such that the first electron beam and the second electron beam are generated in an alternating manner and (b) the control unit is further adapted to control the common deflection unit in a synchronized manner with respect to the control of the electron sources such that there is produced a discrete shift of an active focal spot. Thereby, within a first time period the first focal spot represents the active focal spot and within a second time period the second focal spot represents the active focal spot, respectively. This may provide the advantage that even if the individual electron beam paths are comparatively short, a large discrete focal spot shift can be achieved on the anode surface.

According to a further aspect of the invention there is provided an X-ray system, in particular a medical X-ray imaging system like a computed tomography system. The provided X-ray system comprises an X-ray tube according to any one of the above-described embodiments.

This aspect of the invention is based on the idea that the above-described X-ray tube may be used for various X-ray systems in particular for medical diagnosis.

One may take benefit from illuminating an object under examination with two different sets of X-rays, whereby the two X-ray sets penetrate the object with at least slightly different illumination angles. When using a detector array for sensing the X-rays having traversed the object, one can design the X-ray system such that the so-called interleaving technique is applied. Thereby, neighboring X-rays originating from different focal spots are separated from each other by a distance being half of the distance between neighboring X-rays in the case when only one focal spot is used. This has the advantage that when two X-ray acquisitions being assigned to the two focal spots are combined in an appropriate manner, the spatial resolution of the X-ray system may be enhanced. Under optimal conditions the spatial resolution may be doubled.

A further advantage of the described method can be exploited in computed tomography (CT) when comparatively large objects are examined. By switching the position of the active focal spot in an axial direction with respect to a rotational axis of a CT scanning unit additional projection views may be generated for each view angle of the scanning unit, which scanning unit comprises the X-ray tube and a corresponding X-ray detector. This will allow for employing smaller X-ray detectors without having the disadvantage that for a certain view angle border regions of the object under examination are not located within a cone-shaped or fan-shaped X-ray beam originating from a single focus X-ray tube and impinging onto the X-ray detector.

It has to be mentioned that the described X-ray system may also be used for other purposes than medical imaging. For instance the described X-ray system may also be employed e.g. for security systems such as baggage inspection apparatuses. According to a further aspect of the invention there is provided a method for generating X-rays, in particular for generating X-rays being used for medical X-ray imaging like computed tomography. The provided method comprises using an X-ray tube according to any one of the above-described embodiments of the X-ray tube.

It has to be noted that embodiments of the invention have been described with reference to different subject matters. In particular, some embodiments have been described with reference to apparatus type claims whereas other embodiments have been described with reference to method type claims. However, a person skilled in the art will gather from the above and the following description that, unless other notified, in addition to any combination of features belonging to one type of subject matter also any combination between features relating to different subject matters, in particular between features of the apparatus type claims and features of the method type claims is considered to be disclosed with this application.

The aspects defined above and further aspects of the present invention are apparent from the examples of embodiment to be described hereinafter and are explained with reference to the examples of embodiment. The invention will be described in more detail hereinafter with reference to examples of embodiment but to which the invention is not limited.

### BRIEF DESCRIPTION OF THE DRAWINGS

FIG 1a shows a side view of a multi electron beam X-ray tube comprising a linear arrangement of three electron sources.

FIG. 1b shows a side view of the X-ray tube depicted in FIG. 1a, wherein a common magnetic deflection unit for the electron beams originating from the three electron sources is shown.

FIG. 2 shows a side view of a multi electron beam X-ray tube comprising a structured stacked anode.

FIG. 3 shows a top view of the multi electron beam X-ray tube depicted in FIG. 2.

FIG. 4 shows a further side view of the multi electron beam X-ray tube depicted in FIG. 1b, wherein the magnetic deflection unit can also be seen in a side view.

FIG. 5 shows a simplified schematic representation of a computed tomography (CT) system according to an embodiment of the present invention, wherein the CT system is equipped with a multiple electron beam X-ray tube.

### DETAILED DESCRIPTION

The illustration in the drawing is schematic. It is noted that in different figures, similar or identical elements are provided with the same reference signs or with reference signs, which are different from the corresponding reference signs only within the first digit.

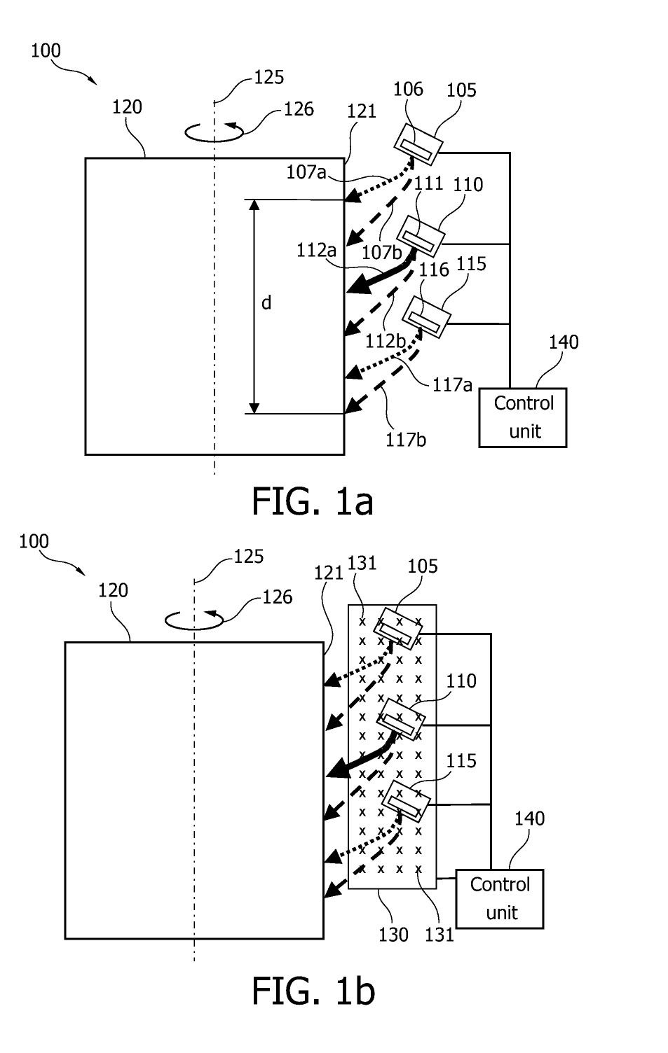

FIG. 1a shows a side view of a multi electron beam X-ray tube 100. The X-ray tube 100 comprises a linear array of three electron sources, a first electron source 105, a second electron source 110 and a third electron source 115. The first electron source 105 comprises an electron emitter filament 106, the second electron source 110 comprises an electron emitter 111 and the third electron source 115 comprises an electron emitter 116. Each of the electron sources 105, 110, 115 is adapted to generate an electron beam projecting along a beam path towards an anode 120.

A common magnetic deflection unit, which is not depicted in FIG. 1a, is used to deflect the generated electron beams 105, 110, 115. Depending on the intensity and the direction of the corresponding magnetic field the electron beam are deflected more or less from their original beam direction. The magnetic field is oriented perpendicular to the plane of drawing. In FIG. 1a there are indicated two exemplary beam paths for each electron source, one beam path corresponds to a maximum magnetic field and the other beam path corresponds to a minimum magnetic field. Thereby, a minimum magnetic field may also be a magnetic field having the same absolute maximal strength but being oriented in an opposite direction with respect to the maximum magnetic field.

Specifically, the first electron beam path 107a indicates the spatial beam propagation of the electron beam originating from the first electron source 105 when the magnetic deflection unit provides a maximum magnetic field. The first electron beam path 107b indicates the corresponding electron beam in the presence of a minimum magnetic field. Accordingly, a second electron beam path 112a corresponds to the beam originating from the second electron source 110, when the deflection unit generates a maximum field. A second electron beam path 112b corresponds to the beam originating from the second electron source 110, when the deflection unit generates a minimum magnetic field. FIG. 1a shows the X-ray tube 100 in an operational state, wherein the second electron source 110 is active and the deflection unit generates a maximum magnetic field. Therefore, the second electron beam path 112a is depicted with a bold arrow indicating the propagation of a second electron beam 112a.

A third electron beam path 117a corresponds to the spatial propagation of an electron beam originating from the third electron source 115, when the deflection unit generates a maximum field. A third electron beam path 117b corresponds to the electron beam originating from the third electron source 115, when the deflection unit generates a minimum magnetic field.

The anode 120 comprises a flat surface 121. Therefore, depending on the temporal activation of the electron sources 105, 110, 115 and on the temporal variation of the magnetic field, a continuously moving focal spot on the anode surface 121 can be generated. For an appropriate activation of the electron sources 105, 110, 115, a control unit 140 is provided, which is coupled to each of the electron sources 105, 110, 115.

According to the embodiment described here the electron sources 105, 110, 115 are operated in a synchronized manner with respect to the common magnetic deflection unit. Thereby alternating electron beams are generated, which effect a quasi-continuous focal spot shift over a comparatively large distance d, which is indicated in FIG. 1a. Thereby, the focal spot can be shifted along a comparatively long distance. By contrast to a single electron beam X-ray tube such a large focal spot shift distance can be achieved by means of the described multi electron beam X-ray tube 100 with much shorter electron drift paths, because each electron source 105, 110, 115 is spatially separated from a corresponding focal spot portion on the anode surface 121 only with a comparatively small distance. Therefore, defocusing and other deteriorating effects regarding the quality of the electron beam can be kept within small limits.

According to the embodiment described here, the anode 120 is a rotational anode capable of rotating around a rotational axis 125. The corresponding rotary motion is indicated by the arrow 126.

FIG. 1b shows also a side view of the multi electron beam X-ray tube 100. By contrast to FIG. 1a, also the common deflection unit 130 is depicted. The common deflection unit 130 generates a magnetic field, which is oriented perpendicular to the plane of drawing. Therefore, the magnetic field is denoted with crosses 131, which indicate that the magnetic field vectors are directed from above the plane of drawing to below the plane of drawing.

The magnetic field 131 has a uniform strength at least within a region covering all electron sources and at least a portion of each electron beam path 107a, 107b, 112a, 112b, 117a, 117b. According to the embodiment described here, such a homogeneous magnetic field is generated by means of a double magnetic yoke. Thereby, one yoke is arranged below the plane of drawing whereas the other yoke is arranged above the plane of drawing.

In order to allow for a synchronized operation of the common deflection unit 130 with respect to the electron sources 105, 110, 115, also the magnetic deflection unit 130 is coupled to the control unit 140.

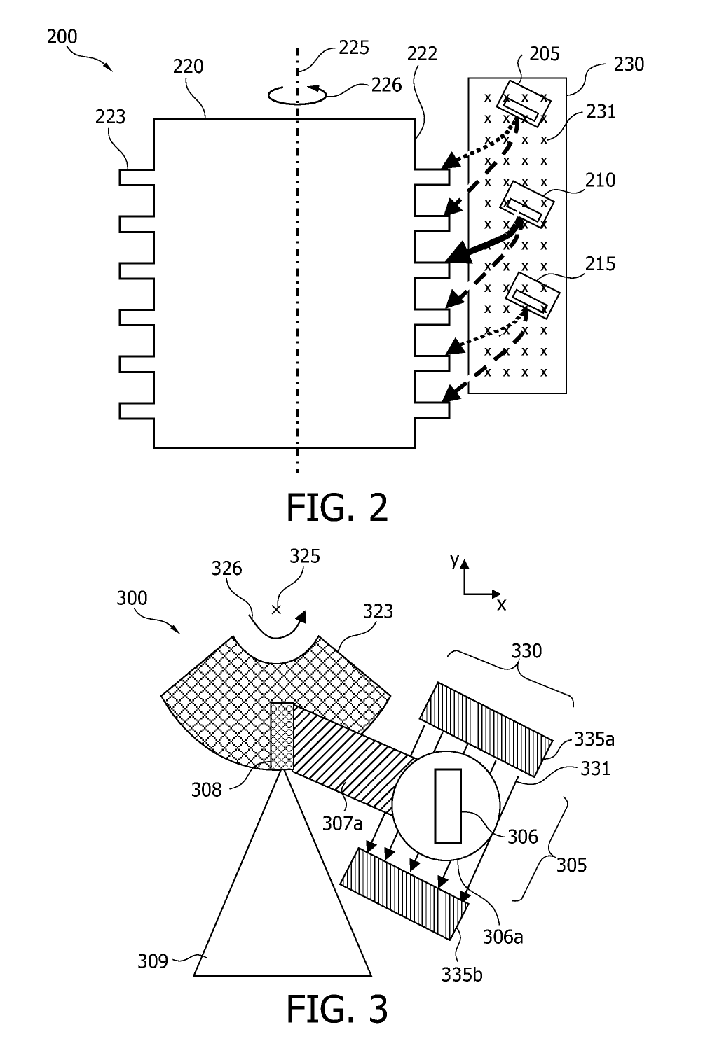

FIG. 2 shows a side view of a multi electron beam X-ray tube 200, which is also equipped with a multiple electron beam generation and deflection unit as has been described above with reference to FIG. 1a and FIG. 1b. Therefore, the X-ray tube 200 comprises three electron sources, a first electron source 205, a second electron source 210 and a third electron source 215. Further, the X-ray tube 200 comprises a common magnetic deflection unit 230, which is adapted to deflect the electron beams by means of a temporal varying magnetic field 231.

By contrast to the embodiment described with reference to FIGS. 1a and 1b, the multi electron beam X-ray tube 200 comprises an anode 220, which has a structured anode surface 222. The cross sections of anode blades 223 protruding from the anode can be seen. Each anode blade 223 represents predetermined focal spot region, whereon one of the deflected electron beam originating from the electron sources 205, 210, 215 can be directed.

In this context it has to be mentioned, that an upper surface of the blades 223 may be cone shaped and angulated with respect to a plane being oriented perpendicular to a rotational axis 225. The corresponding rotary motion is indicated with the arrow 226. Preferably, this angle is selected such that the generated focal spots have the shape of an elongated rectangle. Since the X-rays generated within the focal spot are emitted in a radial direction outward from the rotational axis 225, the projection of the focal spot perpendicular to the direction of the emitted X-rays is much smaller thus leading to a comparatively small focal spot size, which in turn increases the sharpness of X-ray projection images. Preferably, in this projection the focal spots have the shape of a square.

As can further be seen from FIG. 2, there are respectively two protrusions 223 assigned to each of the electron sources 205, 210, 215. This means that there are two predetermined focal spots for each electron source 205, 210, 215. Therefore, the corresponding electron beams can be directed selectively to one of two blades 223. In other words, when all electron beams are activated, a comb structure of active focal spots can be toggled between (a) a first operational state, wherein the electron beams impinge on the first, the third and the fifth blade 223, and (b) a second operational state, wherein the electron beams impinge on the second, the fourth and the sixth blade 223. Thereby, the first blade 223 is the uppermost blade 223 and the sixth blade 223 is the lowermost blade 223 depicted in FIG. 2.

It has to be mentioned that there are of course other ingenious operational states possible. For instance the three electron sources are activated sequentially and the deflection unit 230 is operated in a synchronized manner such that at one time there is only one focal spot active, whereby the focal spot sequentially moves downward by discretely jumping from one blade 223 to the next blade 223 starting from the uppermost blade 223 and ending with the lowermost blade 223.

FIG. 3 shows a top view of the multi electron beam X-ray tube 200 depicted in FIG. 2, which is now denoted with reference numeral 300. In the top view only the uppermost first electron source 305 can be seen. The electron source 305 comprises an electron emitter 306, such as a filament, being surrounded by an electrostatic focusing cup 306a such as a Wehnelt cylinder. The electron source 305 generates a first electron beam 307a projecting onto the uppermost protrusion 323 of the structured anode, which cannot be seen in FIG. 3. Onto the anode blade 323 there is generated a focal spot 308, which represents the origin of a first X-ray beam 309 being generated by the multiple electron beam X-ray tube 300. The focal spot 308 has the shape of an elongated rectangle being oriented radial with respect to a rotational axis 325 of the anode blade 323. The corresponding rotational movement is indicated by the arrow 326.

The first electron beam 307a has a rectangular shape. Its long side is directed radially outward. This causes that the focal spot has a shape corresponding to an elongated rectangle. As has already been explained above, this has the advantage that in a projection of the focal spot along the optical axis of the X-ray beam 309, the elongated focal spot has the shape of a square. Of course, this holds only if the surface of the blade 323 is cone shaped and angulated with respect to the plane of drawing. Thereby, on the one hand a comparatively large area of the blade 323 is illuminated such that a given thermal load of the electron beam 307a is distributed within a comparatively large area. On the other hand the effective focal spot size in the direction of the X-ray beam 309 is comparatively small such that the sharpness of X-ray projection images obtained with the X-ray source 300 is comparatively big.

In order to selectively deflect the electron beam 307a perpendicular to the plane of drawing, a common deflection unit 330 generates a varying magnetic field 331. This field 331, which includes a right angle with the rotational axis 325, is generated in between a first magnetic yoke 335a and a second magnetic yoke 335b. These yokes 335a and 335b represent a magnetic double yoke extending perpendicular to the plane of drawing.

The electron source 305 and the magnetic yokes 335a and 335b are positioned clear off the X-ray beam 309. Therefore, the path of the electron beam 307a is angulated with respect to a horizontal x-direction, to a vertical y-direction and with respect to a z-direction. Thereby, the z-direction is oriented perpendicular to both the x-direction and the y-direction.

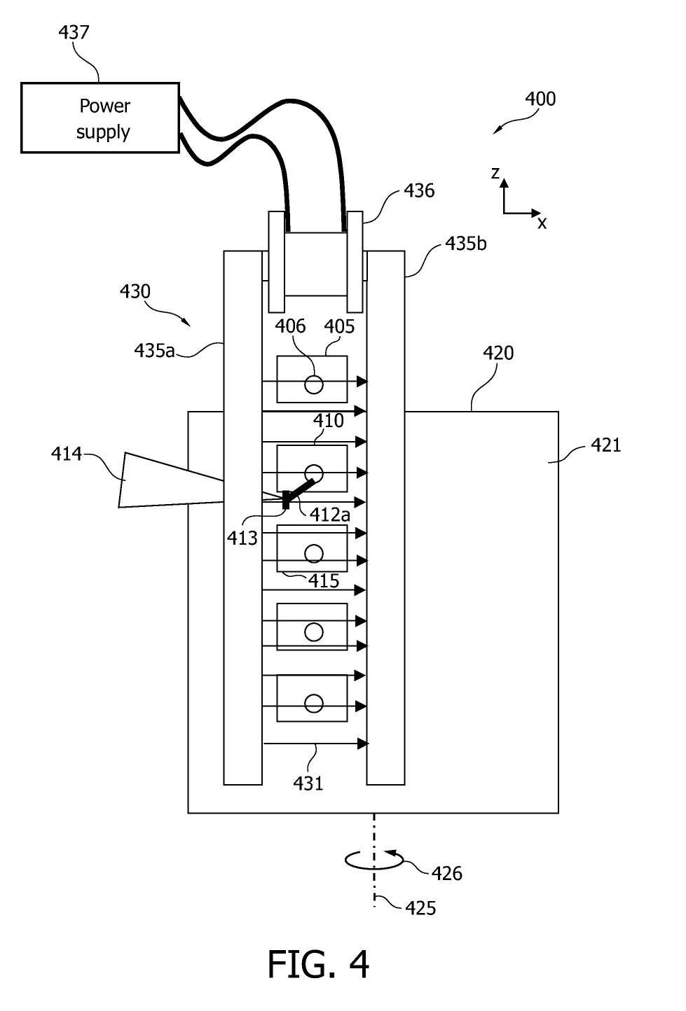

FIG. 4 shows a side view of the multi electron beam X-ray tube 100 depicted in FIG. 1b, which is now denoted with reference numeral 400. The X-ray tube 400 comprises a plurality of electron sources, which are aligned within a linear array. Only the three uppermost electron sources 405, 410 and 415 are denoted with reference numerals. Each of the electron sources comprises an electron emitter filament 406.

FIG. 4 shows the X-ray tube 400 in an operational state, wherein the second electron beam 412a originating from the second electron source 410 is active. The second electron beam 412a generates a focal spot 413 on the flat surface 421 of the anode 420. The focal spot 413, which has again the shape of an elongated rectangle, represents the origin of an X-ray beam 414. The anode 420 is adapted to rotate around a rotational axis 425. The corresponding rotational movement is indicated with the arrow 426. The common magnetic defection unit 430 is used for deflecting the electron beam 412a perpendicular to both (a) the actual propagation direction of the electron beam 412a and (b) the direction of the magnetic field 431. The magnetic field 431 is generated by the first magnetic yoke 435a and the second magnetic yoke 435b. The two magnetic yokes 435a, 435b represent an U-shaped magnetic double yoke. Thereby, the magnetic induction is generated by a solenoid 436, which is fixed in the connecting portion of the magnetic double yoke. The solenoid 436 causes a magnetization of the two magnetic yokes 435a, 435b. The necessary current for the solenoid 436 is provided by a power supply 437 being electrically connected with the solenoid 436.

In the following there will be briefly explained an exemplary operation of the multi electron source X-ray tube 400. When the X-ray tube is switched on, an individual electron source emits an electron beam. The electron beam is deflected by the common magnetic deflection unit 430. The local magnetic field generated by the deflection unit 430 steers the electron beam thus defining the beam path of the electron beam.

When the electron sources are switched on and off in a proper sequence and when the coil is powered accordingly, a continuous flux of electrons is created along the anode surface 421 or along focal spot elements of the anode surface 421, which focal spot elements are not depicted in FIG. 4. Thereby, the position of the resulting electron beam varies as desired. With a variation of the electron beam position also the X-ray focal spot moves.

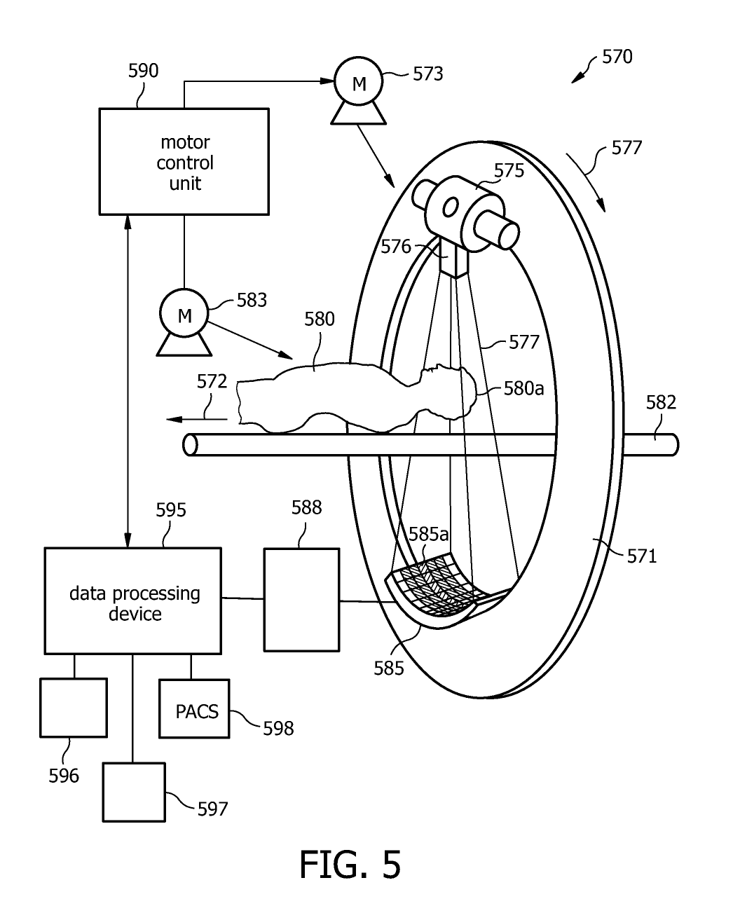

FIG. 5 shows a computer tomography apparatus 570, which is also called a CT scanner. The CT scanner 570 comprises a gantry 571, which is rotatable around a rotational axis 572. The gantry 571 is driven by means of a motor 573.

Reference numeral 575 designates a source of radiation such as an X-ray tube, which emits polychromatic radiation 577. The CT scanner 570 further comprises an aperture system 576, which forms the X-radiation being emitted from the X-ray tube 575 into a radiation beam 107.

The radiation beam 577, which may by a cone-shaped or a fan-shaped beam 577, is directed such that it penetrates a region of interest 580a. According to the embodiment described herewith, the region of interest is a head 580a of a patient 580.

The patient 580 is positioned on a table 582. The patient's head 580a is arranged in a central region of the gantry 571, which central region represents the examination region of the CT scanner 570. After penetrating the region of interest 580a the radiation beam 577 impinges onto a radiation detector 585. In order to be able to suppress X-radiation being scattered by the patient's head 580a and impinging onto the X-ray detector 585 under an oblique angle there is provided a not depicted anti scatter grid. The anti scatter grid is preferably positioned directly in front of the detector 585.

The X-ray detector 585 is arranged on the gantry 571 opposite to the X-ray tube 575. The detector 585 comprises a plurality of detector elements 585a wherein each detector element 585a is capable of detecting X-ray photons, which have been passed through the head 580a of the patient 580.

During scanning the region of interest 580a, the X-ray source 585, the aperture system 576 and the detector 585 are rotated together with the gantry 571 in a rotational direction indicated by an arrow 587. For rotation of the gantry 571, the motor 573 is connected to a motor control unit 590, which itself is connected to a data processing device 595. The data processing device 595 includes a reconstruction unit, which may be realized by means of hardware and/or by means of software. The reconstruction unit is adapted to reconstruct a 3D image based on a plurality of 2D images obtained under various observation angles.

Furthermore, the data processing device 595 serves also as a control unit, which communicates with the motor control unit 590 in order to coordinate the movement of the gantry 571 with the movement of the table 582. A linear displacement of the table 582 is carried out by a motor 583, which is also connected to the motor control unit 590.

During operation of the CT scanner 570 the gantry 571 rotates and in the same time the table 582 is shifted linearly parallel to the rotational axis 572 such that a helical scan of the region of interest 580a is performed. It should be noted that it is also possible to perform a circular scan, where there is no displacement in a direction parallel to the rotational axis 572, but only the rotation of the gantry 571 around the rotational axis 572. Thereby, slices of the head 580a may be measured with high accuracy. A larger three-dimensional representation of the patient's head may be obtained by sequentially moving the table 582 in discrete steps parallel to the rotational axis 572 after at least one half gantry rotation has been performed for each discrete table position.

The detector 585 is coupled to a pre-amplifier 588, which itself is coupled to the data processing device 595. The processing device 595 is capable, based on a plurality of different X-ray projection datasets, which have been acquired at different projection angles, to reconstruct a 3D representation of the patient's head 580a.

In order to observe the reconstructed 3D representation of the patient's head 580a a display 596 is provided, which is coupled to the data processing device 595. Additionally, arbitrary slices of a perspective view of the 3D representation may also be printed out by a printer 597, which is also coupled to the data processing device 595. Further, the data processing device 595 may also be coupled to a picture archiving and communications system 598 (PACS).

It should be noted that monitor 596, the printer 597 and/or other devices supplied within the CT scanner 570 might be arranged local to the computer tomography apparatus 570. Alternatively, these components may be remote from the CT scanner 570, such as elsewhere within an institution or hospital, or in an entirely different location linked to the CT scanner 570 via one ore more configurable networks, such as the Internet, virtual private networks and so forth.

It should be noted that the term “comprising” does not exclude other elements or steps and the “a” or “an” does not exclude a plurality. Also elements described in association with different embodiments may be combined. It should also be noted that reference signs in the claims should not be construed as limiting the scope of the claims.

In order to recapitulate the above described embodiments of the present invention one can state:

It is described an X-ray tube 100, 200 for moving a focal spot within a wide range. The X-ray tube 100, 200 comprises a first electron source 105, which is adapted to generate a first electron beam projecting along a first beam path 107a, 107b, a second electron source 110, which is adapted to generate a second electron beam projecting along a second beam path 112a, 112b and an anode 120, which is arranged within the first beam path 107a, 107b and within the second beam path 112a, 112b such that on a surface 121 of the anode 120 the first electron beam 307a generates a first focal spot 308 and the second electron beam 412a generates a second focal spot 413. The X-ray tube 100, 200 further comprises a common deflection unit 130, 330, 430, which is adapted to deflect the first 307a and the second electron beam 412a, such that the positions of the first 308 and the second focal spot 413 is shifted. The electron sources 105, 110 may be arranged within a linear array allowing for a simple mechanical support of the X-ray sources.

### LIST OF REFERENCE SIGNS:

100 X-ray tube

105 first electron source

106 electron emitter

107a first electron beam path

107b first electron beam path

110 second electron source

111 electron emitter

112a second electron beam path/second electron beam (active)

112b second electron beam path

115 third electron source/further electron source

116 electron emitter

117a third electron beam path

117b third electron beam path

120 anode

121 flat anode surface

125 rotational axis

126 rotary motion

130 common deflection unit/magnetic deflection unit

131 magnetic field

140 control unit

d maximal focal spot shift

200 X-ray tube

205 first electron source

210 second electron source

215 third electron source/further electron source

220 anode

222 structured anode surface

223 protrusion/anode blade

225 rotational axis

226 rotary motion

230 common deflection unit/magnetic deflection unit

231 magnetic field

300 X-ray tube

305 first electron source

306 electron emitter/filament

306a electrostatic focusing cup

307a first electron beam

308 focal spot

309 X-ray beam

323 protrusion/anode blade

325 rotational axis

326 rotary motion

330 common deflection unit/magnetic deflection unit

331 magnetic field

335a magnetic yoke

335b magnetic yoke

400 X-ray tube

405 first electron source

406 electron emitter filament

410 second electron source

412a second electron beam

413 focal spot

414 X-ray beam

415 third electron source

420 anode

421 flat anode surface

425 rotational axis

426 rotary motion

430 common deflection unit/magnetic deflection unit

431 magnetic field

435a magnetic yoke

435b magnetic yoke

436 solenoid

437 power supply

570 medical X-ray imaging system/computed tomography apparatus

571 gantry

572 rotational axis

573 motor

575 X-ray source/X-ray tube

576 aperture system

577 radiation beam

580 object of interest/patient

580a region of interest/head of patient

582 table

583 motor

585 X-ray detector

585a detector elements

587 rotation direction

588 Pulse discriminator unit

590 motor control unit

595 data processing device (incl. reconstruction unit)

596 monitor

597 printer

598 Picture archiving and communication system (PACS)

1. An X-ray tube comprising
a first electron source (105), which is adapted to generate a first electron beam projecting along a first beam path (107a, 107b),
a second electron source (110), which is adapted to generate a second electron beam projecting along a second beam path (112a, 112b),
an anode (120), which is arranged within the first beam path (107a, 107b) and within the second beam path (112a, 112b) such that on a surface (121) of the anode (120) the first electron beam (307a) generates a first focal spot (308) and the second electron beam (412a) generates a second focal spot (413) being separated from the first focal spot (308), and
a common deflection unit (130, 330, 430), which is adapted to deflect the first electron beam (307a) and the second electron beam (412a), such that
the position of the first focal spot (308) and the position of the second focal spot (413) is shifted.

2. The X-ray tube according to claim 1, further comprising
a control unit (140), which
is coupled to the first electron source (105), to the second electron source (110) and to the common deflection unit (130) and which
is adapted to control the first electron source (105), the second electron source (110) and the common deflection unit (130) in a synchronized manner.

3. The X-ray tube according to claim 1, wherein
the anode (120) comprises a first focal spot region and a second focal spot region being at least partially separated from the first focal spot region, whereby
the first focal spot region is assigned to the first electron source (105) and
the second focal spot region is assigned to the second electron source (110).

4. The X-ray tube according to claim 1, wherein
the first electron source (105) is adapted to activate and to deactivate the first electron beam and/or
the second electron source (110) is adapted to activate and to deactivate the second electron beam.

5. The X-ray tube according to claim 1, wherein
the common deflection unit is a magnetic deflection unit (130).

6. The X-ray tube according to claim 5, wherein the magnetic deflection unit (130, 430) is adapted to generate a homogeneous magnetic field (131, 431) having a uniform magnetic field intensity at least within a region covering the first beam path (107a, 107b) and the second beam path (112a, 112b) at least partially.

7. The X-ray tube according to claim 5, wherein
the first electron source (105) and/or the second electron source (110) is made from a non-ferromagnetic material.

8. The X-ray tube according to claim 1, further comprising
a further electron source (115), which is adapted to generate a further electron beam projecting along a further beam path (117a, 117b),
wherein the further electron beam generates a further focal spot on the surface (121) of the anode (120), the further focal spot being separated from the first focal spot and from the second focal spot, and
wherein the common deflection unit (130) is adapted to deflect the further electron beam such that the position of the further focal spot is shifted.

9. The X-ray tube according to claim 8, wherein
the first electron source (105, 405), the second electron source (110, 410) and the further electron source (115, 415) are arranged in a linear array of electron sources.

10. The X-ray tube according to claim 1, wherein
the anode (120) comprises a flat anode surface (121) at least along a direction being defined by the various focal spot positions.

11. The X-ray tube according to claim 2, wherein
the control unit (140) is adapted to control the electron sources (105, 110, 115) such that the first electron beam and the second electron beam are generated in an alternating manner and
the control unit (140) is further adapted to control the common deflection unit (130) in a synchronized manner with respect to the control of the electron sources (105, 110, 115) such that
there is produced a quasi continuous shift of an active focal spot, whereby within a first time period the first focal spot represents the active focal spot and within a second time period the second focal spot represents the active focal spot, respectively.

12. The X-ray tube according to claim 1, wherein
the anode (220) comprises a structured anode surface (223) at least along a direction being defined by the various focal spot positions.

13. The X-ray tube according to claim 2, wherein
the control unit (140) is adapted to control the electron sources (105, 110, 115) such that the first electron beam and the second electron beam are generated in an alternating manner and
the control unit (140) is further adapted to control the common deflection unit (130) in a synchronized manner with respect to the control of the electron sources (105, 110, 115) such that
there is produced a discrete shift of an active focal spot, whereby
within a first time period the first focal spot represents the active focal spot and within a second time period the second focal spot represents the active focal spot, respectively.

14. An X-ray system, in particular a medical X-ray imaging system like a computed tomography system (570), the X-ray system comprising an X-ray tube (100, 200, 575) according to claim 1.

15. A method for generating X-rays, in particular for generating X-rays being used for medical X-ray imaging like computed tomography, the method comprising using an X-ray tube (100, 200, 575) according to claim 1.
